(19) United States
(12) Patent Application Publication
(10) Pub. No.: US 2009/0326294 A1
(43) Pub. Date: Dec. 31, 2009

(54) Reactor Process for Smaller Batch Transfers of Catalyst

(76) Inventors: Douglas G. Stewart, Wheeling, IL (US); Mark G. Riley, Hinsdale, IL (US); Peter M. Bernard, Chicago, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/145,588

(22) Filed: Jun. 25, 2008

(51) Int. Cl. C07C 5/00

(52) U.S. Cl. 585/500

(57) ABSTRACT

A moving bed of catalyst loses activity as it moves through the reactor. Creating multiple passes for the process fluid moving across a catalyst bed, increases the utilization of the catalyst and creates a step-wise counter current flow of catalyst and process fluid, where the catalyst flows in the axial direction of the reactor, and the process fluid flows radially, with step-wise axial direction flow when the flow is reversed to flow back across the catalyst bed. The flow improves the temperature profile of the bed and allows higher temperature fluid contacting the less active catalyst.

### FIELD OF THE INVENTION

This invention relates to a radial flow reactor for use in a hydrocarbon conversion process. The process involves a catalyst moving down through the reactor, where the catalyst becomes deactivated over time, and the fluid reactants move across the reactor bed.

### BACKGROUND OF THE INVENTION

A process for the conversion of paraffins to olefins involves passing a normal paraffin stream over a highly selective catalyst, where the normal paraffin is dehydrogenated to the corresponding mono-olefin. The dehydrogenation reaction is achieved under mild operating conditions, thereby minimizing the loss of feedstock.

The typical process involves the use of a radial flow reactor where a paraffin feedstock is contacted with a dehydrogenation catalyst under reaction conditions. The typical process involves dehydrogenating linear paraffins in the C7 to C11 range to produce olefins used as plasticizers, for dehydrogenating paraffins in the C10 to C14 range to produce linear olefins for the production of linear alkyl benzenes (LABs), and for dehydrogenating paraffins in the C12 to C17 range to produce detergent alcohols or olefin sulfonates.

The process is affected by reactor design, and processing costs can increase substantially if the catalyst is underutilized, the reactor is required to be shut down to reload catalyst, or operating conditions need to be significantly changed as the catalyst deactivates.

### SUMMARY OF THE INVENTION

In a radial flow reactor, the catalyst moves downward through an annular region, while the fluid reactants move across the catalyst bed. As the catalyst moves downward and processes more of the feedstream, it becomes less active. The reduction in activity requires the increase in temperature of the operating conditions to maintain the desired level of conversion. The present invention operates to take advantage of the catalysts declining activity as the catalyst flows through the reactor. The process fluid enters the reactor centerpipe, and the flow is restricted in the centerpipe to direct the process fluid to flow over the catalyst bed. The fluid is further restricted in the outer annular region and redirected to flow back over the catalyst bed, providing a step-wise counter current flow of catalyst and process fluid. To take advantage of the declining catalyst activity as the catalyst flows through the reactor, and the endothermic properties of the reaction, the process fluid is heated to a temperature greater than the temperature for a regular single pass process with fresh catalyst.

Other objects, advantages and applications of the present invention will become apparent to those skilled in the art from the following detailed description and drawings.

### BRIEF DESCRIPTION OF THE DRAWINGS

FIG. 1 is a standard radial flow reactor design;

FIG. 2 is a diagram of one embodiment of the invention, with a single valve;

FIG. 3 is a diagram of a second embodiment with a cone fill for reducing the void space in the centerpipe, and a bypass;

FIG. 4 is a third embodiment of the invention with multiple valves in the centerpipe and multiple passes through the catalyst bed; and

FIG. 5 is a fourth embodiment with a flow bypass.

### DETAILED DESCRIPTION OF THE INVENTION

The use of radial flow reactors is common in the hydrocarbon processing industry. One example is the conversion of paraffins to olefins, where a paraffin rich gas is passed over a catalyst to dehydrogenate the paraffins to generate a product stream comprising olefins. The dehydrogenation reaction is achieved under mild operating conditions to minimize loss of the feedstock to byproducts. The paraffins to olefins conversion is important for the production of linear alkylbenzenes, where linear paraffins in the C7 to C26 range to produce linear alpha olefins.

In some processes, as the catalyst deactivates, the operating temperature is raised to off-set the reduction in catalyst activity, until the selectivity is too poor to continue the process. This is often done 30-40 times during a cycle, and then the catalyst is replaced in the reactor. Before the process of replacing catalyst begins, the reactor operating temperature is lowered to accept the cooler catalyst, and the feed flow is reduced to insure that catalyst is not pinned to the catalyst screen. That is, at sufficiently high flow rates of the reactor fluid, the catalyst can be held against the catalyst screen and not flow through the reactor, and therefore to insure its movement, the fluid flow is reduced. This process requires a significant amount of time and results in lost productivity.

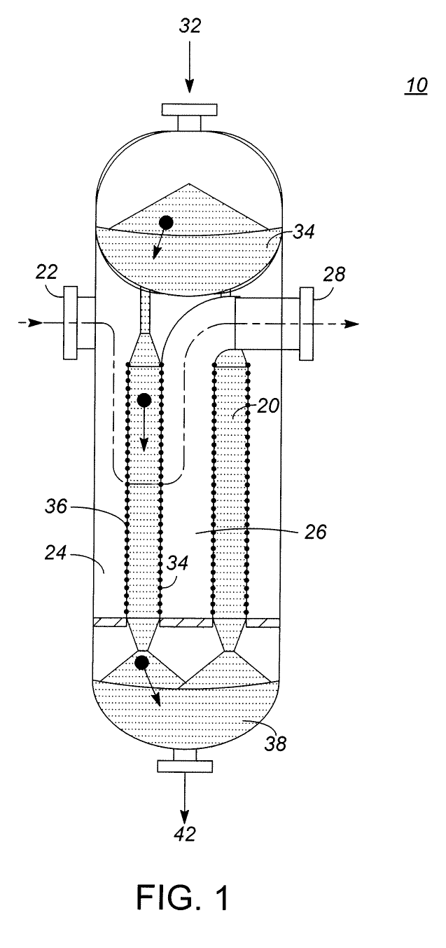

A radial flow reactor 10, as shown in FIG. 1 and as used in a paraffins to olefins conversion reactor, comprises an annular reactor bed 20 for holding catalyst, an inlet port 22 for admitting a fluid, where the fluid flows around an annular space 24, across the reactor bed 20, and into a centerpipe 26. The product then flows out the centerpipe 26 to a product outlet port 28. Fresh catalyst enters through a catalyst inlet port 32, into a reduction zone 34, where the catalyst is prepared with hot hydrogen before entry to the reactor bed 20. Catalyst enters the reactor bed 20, where it is confined between two annular screens 34, 36, flows through the reactor bed 20 and is collected in a collection zone 38. The spent catalyst is withdrawn from the collection zone 38 through a catalyst outlet port 42 and directed to a regeneration unit.

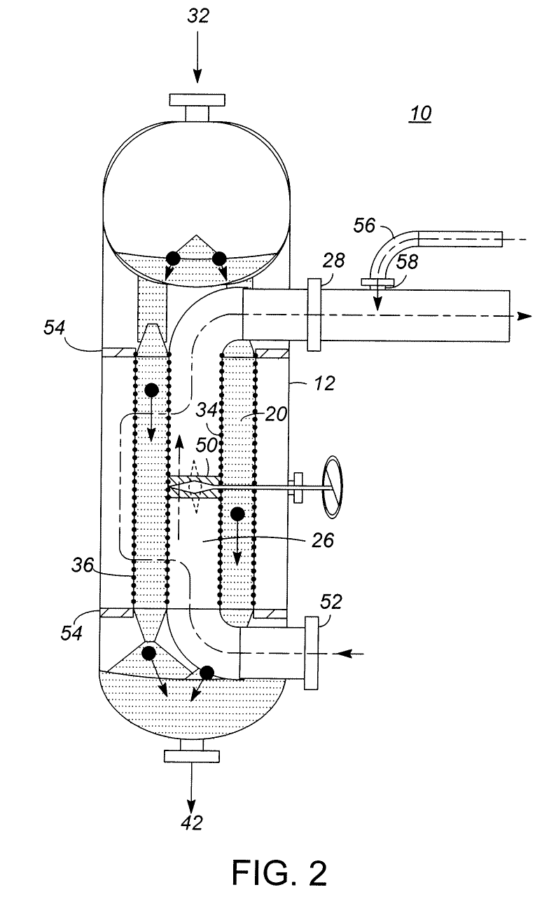

The present invention allows for the catalyst to be added in smaller increments, and to increase the catalyst utilization. The invention forces multiple passes of the fluid through the annular reactor bed 20. In a first embodiment, as shown in FIG. 2, the radial flow reactor comprises a substantially cylindrical housing 12 having a central axis, and having a catalyst inlet port 32 at the top of the reactor 10 and a catalyst outlet port 42 at the bottom of the reactor 10. The reactor 10 includes a centerpipe 26, which can comprise a perforated tube, a tubular structure with catalyst screens 34, or any other structure that permits the flow of fluid across the centerpipe wall 34 which is also a catalyst screen, while preventing the flow of catalyst into the centerpipe 26. The reactor 10 further includes an annular perforated screen 36 disposed between the centerpipe wall 34 and the housing 12, and a restriction 50 disposed within the centerpipe 26. The fluid inlet 52 is now in fluid communication with one end of the centerpipe 26. The restriction 50 forces the fluid across the reactor bed 20, and the fluid returns through the reactor bed 20 at a position further up the reactor bed 20 and returns to the centerpipe 26. The reactor 10 further includes seals 54 between the annular screen 36 and the reactor housing 12.

In one embodiment, the restriction 50 is a valve that can be opened to allow free passage of the fluid, or closed to force the fluid through the reactor bed 20. With a valve, the restriction 50 can allow some by-pass flow that does not go through the catalyst bed 20. This is useful for reducing the flow rate when there is catalyst pinning, or there is a need to control or reduce the amount of reaction taking place. The reactor 10 further can optionally include a quench fluid line 56. The quench fluid line 56 is in fluid communication with the centerpipe outlet 28, through a quench fluid inlet port 58. The quench fluid can be used during any by-pass operation to facilitate maintaining a stable operation of the reactor 10. The quench fluid, usually gaseous hydrogen, would cool the outlet stream when there is insufficient endothermic reaction taking place in the reactor bed 20, or when there is a significant bypass of the fluid from going across the reactor bed 20. An alternate quench fluid is a liquid paraffin, entering through the quench inlet port 58 at a temperature of less than 100° C.

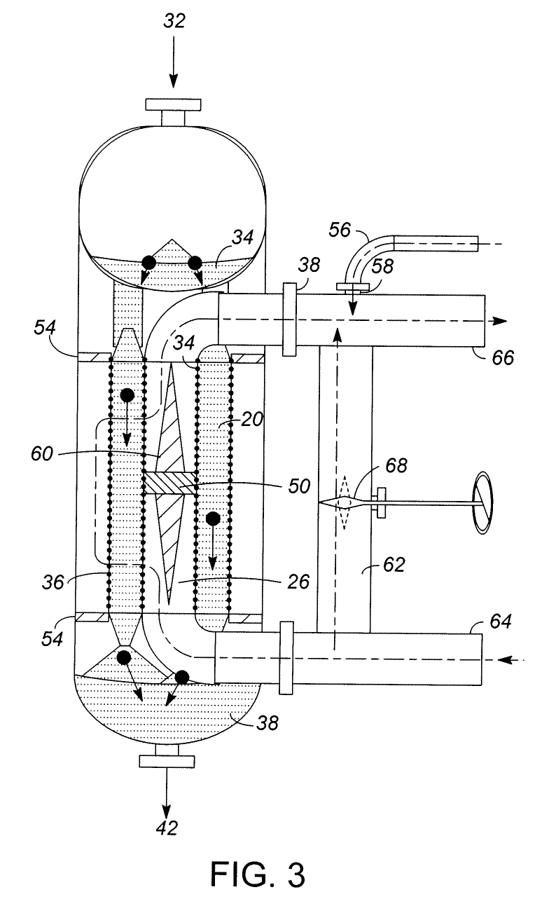

In a second embodiment, as shown in FIG. 3, the restriction 50 is a seal. A seal provides a simpler structure for the reactor 10, and removes a need for providing ports through the reactor for the mechanical components required to open and close the valve. In this embodiment, an optional volume fill section 60 in the centerpipe 26. The volume fill section 60 reduces the residence time of the fluid in the centerpipe 26, which can reduce the chance of side reactions, such as thermal cracking taking place. Preferably, the volume fill section 60 is a conic shaped section, and provides additional control over directing flow through the reactor bed 20 and directing the product stream out of the centerpipe 26. The second embodiment further includes an optional by-pass conduit 62. The by-pass conduit 62 allows for control of flow through the reactor 10, and can reduce the flow when there is pinning of catalyst, or for other reasons where it is necessary to reduce the flow to the reactor 10. The by-pass conduit 62 provides fluid communication between the inlet pipe 64 to the centerpipe 26 and the outlet pipe 66 from the centerpipe 26. The by-pass conduit 62 includes a valve 68 for opening and closing the by-pass conduit 62.

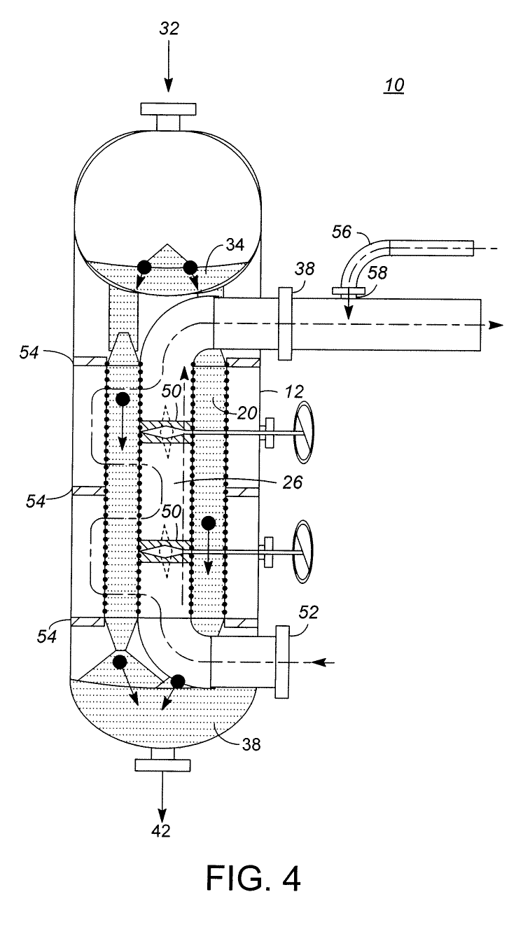

In a third embodiment, as shown in FIG. 4, the reactor 10 comprises a plurality of restrictions 50 within the centerpipe 26. In FIG. 4 two restrictions 50 are shown for illustration, but more restrictions 50 can be added. The number of restrictions 50 is subject to the size of the reactor 10, the length of the centerpipe 26, and other design considerations, such as limits on operating conditions and mechanical limitations. The reactor 10 includes seals 54 at the top and bottom of the reactor bed 20 between the annular perforated screen 36 and the reactor housing 12. The reactor 10 includes additional seals 54 positioned between successive restrictions 50. This provides for a plurality of passes in the reactor bed 20 by the process fluid. When the restrictions 50 are valves, the valves provide control to by-pass the reactor bed 20 when there is catalyst pinning or the need to reduce the rate of reaction.

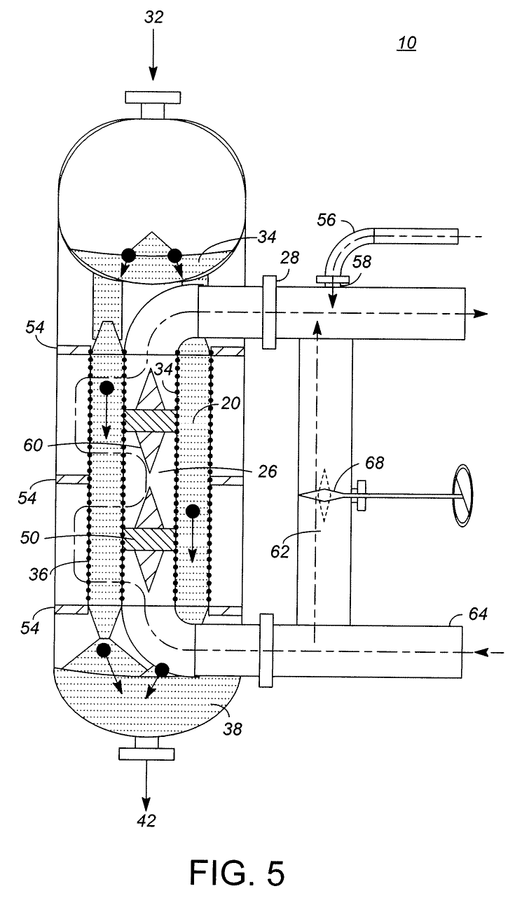

A fourth embodiment, as shown in FIG. 5, comprises a reactor 10 with a plurality of restrictions 50 within the centerpipe 26, wherein the restrictions 50 are seals that do not open or close, as described above in the second embodiment. The centerpipe 26, optionally includes volume fill sections 60 for reducing residence time of the fluid within the centerpipe. This embodiment further includes an optional by-pass conduit 62. The by-pass conduit 62 allows for control of flow through the reactor 10. The by-pass conduit 62 provides fluid communication between the inlet pipe 64 to the centerpipe 26 and the outlet pipe 66 from the centerpipe 26. The by-pass conduit 62 includes only one valve 68 for opening and closing the by-pass conduit 62, instead of multiple valves as in the third embodiment.

By providing two or more passes of the fluid across the reactor bed 20, and with the flow of fluid crossing the catalyst having the longest reactor residence time first, and the newest catalyst last, lower activity catalyst contacts higher temperature gas and improves the process. The catalyst is added in a semi-continuous process in small batches that has cooler catalyst added to the top of the reactor bed 20 where the reactor is coolest, and the catalyst withdrawn from the catalyst bed is the hottest and has lost the most activity.

The reactor 10 with multiple passes provides a pseudo counter-current radial flow reactor. The process fluid enters the reactor centerpipe 26, and successively contacts higher activity catalyst as the process fluid passes back and forth across the reactor bed 20, before exiting the reactor 10 as a product stream. The pseudo counter-current radial flow reactor also provides a favorable temperature profile, by allowing hotter gas to enter the reactor and contact lower activity catalyst. As the process fluid proceeds through the reactor and reacts, the temperature drops and the process fluid contacts successively the higher activity catalyst, and reduces the incremental batch feed of catalyst.

The present invention is a process for increasing the use of catalyst flowing through a radial flow reactor. The process involves controlling the flow of catalyst and process fluid to improve the yields and to reduce the amount of heating and cooling of the reactor when catalyst is added to the reactor. One process that uses a radial flow reactor is a paraffin dehydrogenation process. The reaction conditions include pressure between 1 kPa and 1013 kPa, and a temperature between 400° C. and 900° C., with the temperature preferably in the range 400° C. to 500° C. The reaction is endothermic and the catalyst and the reactants in the reactor are cooled as the reaction proceeds. As the reaction proceeds over time, the catalyst deactivates, and in order to keep the yields up, the temperature of the reactor is heated. The process fluid can also be heated an additional 10° C. to 20° C. over the initial feedstock temperature to the reactor as the catalyst ages and deactivates.

By flowing the process fluid across the catalyst multiple times, the catalyst can be better utilized and yields increased. This is carried out by passing the process fluid over the catalyst with the longest residence time, or most deactivated catalyst, in the reactor first, and then redirecting the flow of the process fluid over the catalyst bed having successively less deactivation. For a radial flow reactor, instead of the fluid entering the outer annular region, and exiting the centerpipe, or the reverse, the flow enters the centerpipe, flows across the catalyst bed, is redirected in the outer annular region, flows back across the catalyst bed, and the return flow is directed out the upper end of the centerpipe. The flow is restricted in the centerpipe to prevent the process fluid bypassing the catalyst bed, and the restriction, or the flow can be controlled to prevent pinning of the catalyst as it flows through the reactor. The process can be repeated for multiple passes of the process fluid over the catalyst bed providing a step-wise countercurrent flow of the process fluid with the catalyst flowing through the reactor, with the catalyst flowing in the axial direction of the reactor, and the process fluid flowing radially across the catalyst, but also flowing in a step-wise axial direction.

The process can further include heating the process fluid to a temperature greater than the normal operating temperature for this process. The added heating compensates for the loss of activity of the catalyst as it progresses through the reactor, and to compensate for the loss of heat due to the reaction being endothermic.

The process can include treating the catalyst in a reduction zone before feeding the catalyst to the reactor. The catalyst is treated with a hot hydrogen gas, where the gas is at a temperature between 350° C. to 500° C. The catalyst, after passing through the reactor, is collected and passed to a hydrogen stripping zone for removal of heavy hydrocarbons that accumulated on the catalyst during the process.

While the invention has been described with what are presently considered the preferred embodiments, it is to be understood that the invention is not limited to the disclosed embodiments, but it is intended to cover various modifications and equivalent arrangements included within the scope of the appended claims.

1. A method for reducing the size of batch transfer of catalyst through a reactor for processing hydrocarbons, comprising:
transferring the catalyst transfer at smaller more frequent intervals;
restricting the flow of the process fluid through the centerpipe;
directing the flow of the process fluid from the centerpipe through the annular catalyst bed to an outer annular space to react at reaction conditions;
redirecting the flow of the process fluid from the outer annular space through the annular catalyst bed to react at reaction conditions, thereby creating a return flow; and
directing the return flow of the process fluid out of the reactor.

2. The method of claim 1 further comprising redirecting the return flow of the process fluid back through the annular bed multiple times.

3. The method of claim 1 wherein the reactor is a radial flow reactor, and the process is an endothermic reaction.

4. The method of claim 3 further comprising heating the process fluid to a temperature greater than that for the process with fresh catalyst.

5. The process of claim 4 wherein the process fluid temperature is raised 10° C. to 20° C.

6. The method of claim 1 further comprising;
treating the catalyst with hot hydrogen gas before feeding the catalyst into the reactor; and
feeding the catalyst in a continuous process to the reactor.

7. The method of claim 6 wherein the hot hydrogen gas is at a temperature from 350° C. to 500° C.

8. The method of claim 1 further comprising:
collecting the catalyst from the bottom of the reactor; and
passing the catalyst to a hydrogen stripping zone for removal of heavy hydrocarbons from the catalyst.

9. The method of claim 1 wherein reaction conditions include a temperature between 400° C. and 900° C.

10. The method of claim 1 wherein the reaction is an endothermic dehydrogenation reaction.

11. A process for increasing the use of catalyst in hydrocarbon conversion, when the catalyst deactivates overtime, comprising:
flowing the catalyst continuously downward through a catalyst bed, wherein the catalyst residence time increases with distance traveled through the catalyst bed;
flowing a hydrocarbon stream across the oldest catalyst first to react at reaction conditions;
redirecting the hydrocarbon stream across the catalyst bed of successively younger catalyst to react at reaction conditions, thereby generating a product hydrocarbon stream; and
withdrawing the product hydrocarbon stream from the top of the reactor.

12. The method of claim 11 wherein the catalyst flows through a radial flow reactor in the axial direction and the process fluid flows in the radial direction.

13. The method of claim 11 wherein the reaction conditions include a temperature between 400° C. and 900° C., and a pressure between 1 kPa and 1013 kPa.

14. The process of claim 11 wherein the reaction is an endothermic reaction, and the process further comprises heating the hydrocarbon feedstream to a temperature greater than a feedstream temperature with fresh catalyst.
